(19) United States
(12) Patent Application Publication
(10) Pub. No.: US 2008/0045908 A1
(43) Pub. Date: Feb. 21, 2008

(54) Medical device including a metallic tube fillet welded to a core member

(75) Inventors: Ric Gould, Brooklyn Park, MN (US); Mort Yazdanpanah, Minnetonka, MN (US); Dave B. Johnson, Hopkins, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc.

(21) Appl. No.: 11/504,833

(22) Filed: Aug. 16, 2006

(51) Int. Cl. A61M 5/32

(52) U.S. Cl. 604/272

(57) ABSTRACT

A medical device, such as a guidewire, or the like, that includes an elongated metallic tubular member fillet welded to a core member. For example, the tubular member may define an inner lumen and include an end, and a metallic core member includes a first portion disposed within the lumen and a second portion extending from the end of the tubular member, and the end of the metallic tubular member is attached to the outer surface of the core member with a fillet weld and/or a weld having a generally triangular and/or ramp-like cross-sectional shape. Methods of creating such a weld and/or making a medical device including such structure are also disclosed.

### FIELD OF TECHNOLOGY

The invention relates generally to medical devices. More specifically, the invention relates to an intracorporal medical device, such as a guidewire, or the like, including a metallic tubular member disposed about and attached to a core member.

### BACKGROUND

The use of intravascular medical devices has become an effective method for treating many types of vascular disease. In general, one or more suitable intravascular devices are inserted into the vascular system of the patient and navigated through the vasculature to a desired target site. Using this method, virtually any target site in the patient's vascular system may be accessed, including the coronary, cerebral, and peripheral vasculature. Examples of therapeutic purposes for intravascular devices include percutaneous transluminal angioplasty (PTA) and percutaneous transluminal coronary angioplasty (PTCA).

When in use, intravascular devices, such as a guidewire, may enter the patient's vasculature at a convenient location and then can be urged to a target region in the anatomy. The path taken within the anatomy of a patient may be very tortuous, and as such, it may be desirable to combine a number of performance features in the intravascular device. For example, it is sometimes desirable that the device have a relatively high level of pushability and torqueability, but also include a desired level of flexibility, particularly near its distal end, for example, to aid in navigation. In that regard, some medical devices incorporate the use of a metallic tubular member disposed about and/or attached to a core member to achieve certain desirable characteristic. However, attaching the metallic tubular member to a core member in a desirable manner can sometimes be problematic.

A number of different elongated medical device structures, assemblies, and methods are known, each having certain advantages and disadvantages. However, there is an ongoing need to provide alternative elongated medical device structures, assemblies, and methods. In particular, there is an ongoing need to provide alternative medical devices including the metallic tubular member disposed over a core member to provide for desirable characteristics, and/or alternative structures or methods for attaching a metallic tubular member to a core member.

### SUMMARY OF SOME EMBODIMENTS

The invention provides several alternative designs, materials and methods of manufacturing and using alternative elongated medical device structures and assemblies.

Some example embodiments relate to a medical device, such as a guidewire, or the like, that includes an elongated metallic tubular member defining an inner lumen and including an end, and a metallic core member including a first portion disposed within the lumen of the tubular member, and a second portion extending from the end of the tubular member, wherein the end of the metallic tubular member is attached to the outer surface of the core member with a fillet weld and/or a weld having a generally triangular and/or ramp-like cross-sectional shape. In some embodiments, the tubular member includes an outer diameter that is greater than the outer diameter of at least a section of the core member, and the weld can provide a tapered and/or ramp like transition between the two different outer diameters. For example, in some embodiments, a corner having an interior angle may be formed between the end of the tubular member and the outer surface of the core member, and weld metal and/or material is deposited in the corner and may provide such a tapered transition. Methods of creating such a weld and/or making a medical device including such structure are also disclosed.

The above summary of some embodiments is not intended to describe each disclosed embodiment or every implementation of the present invention. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial longitudinal cross sectional view of one embodiment of a guidewire including a core wire and a metallic tubular member fillet welded to the core;

FIG. 2 is a partial cross-sectional view of the guidewire of FIG. 1, showing the start of fillet welding the proximal end of the metallic tubular member to the core;

FIGS. 3 is a partial cross-sectional view of the guidewire shown in FIG. 1 showing the completion of fillet welding the proximal end of the metallic tubular member to the core; and

FIG. 4 is a partial longitudinal cross sectional view of another embodiment of a guidewire including a core member and a metallic coil member fillet welded to the core.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

As will be appreciated, at least some embodiments relate to a medical device that includes a metallic tubular member disposed about and attached to a metallic core member. Medical devices incorporating such a structure could be guidewires or catheters or other such medical devices.

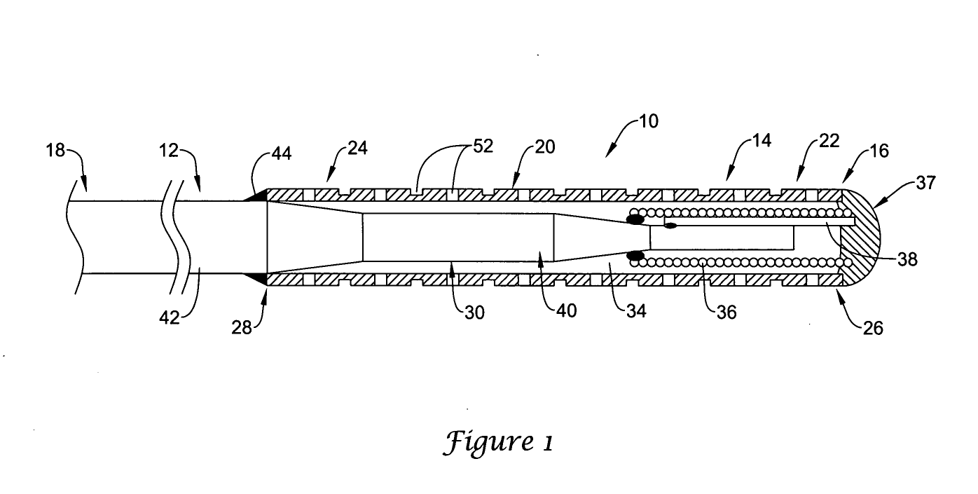

Refer now to FIG. 1, which illustrates a medical device 10 in accordance with one example embodiment. In the embodiment shown, the medical device 10 is in the form of a guidewire 10. Guidewire 10 can include a proximal region 12, a distal region 14, a distal end 16, and a proximal end 18. As used herein, the proximal region 12 and the distal region 14 may generically refer to any two adjacent guidewire sections along any portion of the guidewire 10. The guidewire 10 includes a generally tubular member 20 that includes a distal section 22, a proximal section 24, a distal end 26, and a proximal end 28. The tubular member 20 includes an inner lumen 34, and may extend longitudinally along a longitudinal axis. The tubular member 20 may include a plurality of slots 52 formed therein, for example, to provide a degree of lateral flexibility while maintaining a degree of torque transmission ability. Some additional aspects of the tubular member 20 will be discussed in more detail below.

A distal tip member 37 may be disposed at the distal end 26 of the tubular member 20 and/or the distal end 16 of the guidewire 10. The distal tip member 37 may be any of a broad variety of suitable structures, for example, a solder tip, a weld tip, a pre-made or pre-formed metallic or polymer structure, or the like, that is attached or joined to the distal end of the tubular member 20 using a suitable attachment technique.

The guidewire 10 may also include a core member 30 that may be attached to the tubular member 20, and extend from a location within the tubular member 20 and/or from the proximal end 28 of the tubular member 20, for example, to the proximal end 18 of the guidewire 10. As can be appreciated, a portion of the core member 30 may extend into at least a portion of the lumen 34. In the embodiment shown, the core member 30 includes a distal portion 40 that extends within the lumen 34, and a proximal portion 42 that extends proximally from the tubular member 20. In the embodiments shown, the core member 30 ends proximally from the distal tip member 37 and/or proximally of the distal end 26 of the tubular member 20. In other embodiments, however, core member 30 may extend to, and be attached to the distal tip member 37.

The guidewire 10 may also include other structures, such as such as a shaping wire or ribbon, one or more coils, marker members, or the like, or others, but such structures are not necessary in some other embodiments. In the embodiment shown, the guidewire 10 includes a distal coil member 36 and a shaping ribbon member 38 that may be, for example, attached to and extend distally from the distal end of the core wire 30, and may be attached, for example, to the tip member 37. The materials used for such structures can be any that are suitable for their intended purpose. Some example materials are discussed below. Additionally, the attachment of the various components can be achieved using any suitable attachment techniques, some examples of which may include adhesive bonding, welding, soldering, brazing, mechanical bonding and/or fitting, or the like, or any other suitable technique.

In at least some embodiments, however, an end, such as the proximal end 28, of the tubular member 20 can be attached to the core member 30 with a “fillet weld” 44. In some cases a fillet weld (pronounced “FILL-it,” not “fil-LAY”) can be characterized as a weld used to make lap joints, corner joints, and T joints. The fillet weld 44 may be roughly and/or generally triangular and/or ramp-like and/or wedge-like in cross-section, although its shape is not always a right triangle or an isosceles triangle, or not necessarily an exact triangle. For example, one or more of the sides may be curvilinear and/or ramp-like. In making a fillet weld, weld metal can be deposited in a corner formed by the fit-up of the two members (for example, the core member 30 and the tubular member 20) and can penetrate and fuse with the base metals of the two members to form the joint. Note that for the sake of clarity, the drawings do not show the penetration of the weld metal, but such penetration may, and in fact, is likely to exist. The use of such a fillet weld 44 may provide for certain advantages in some embodiments.

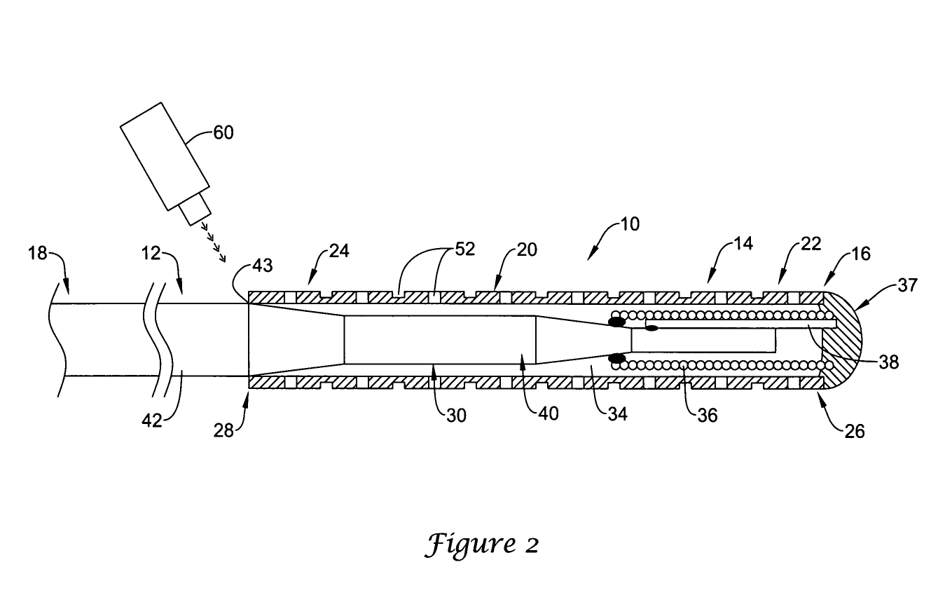

For example, refer now to FIG. 2, which shows the guidewire 10 just prior to the attachment of the proximal end 28 of the tubular member 20 to the core wire 30 with a fillet weld 44. As can be appreciated, the core member 30 can include an outer surface, and can include at least a section having an outer diameter that is less than the outer diameter of the tubular member 20. As such, when the tubular member 20 is disposed about the core member 30, this relative difference in outer diameter between the two members can create a rather abrupt change in the outer diameter of the guidewire 10. For example, an interior angle and/or corner 43, which may be rather aggressive, may be defined between the outer surface of the core wire 30 and the proximal end 28 of the tubular member 20. Such an aggressive transition in the outer diameter of the guidewire 10 may present certain problems. For example, in some cases, other devices that are guided over the guidewire may catch on and/or be damaged by such an aggressive transition. Additionally, an abrupt change in the flexibility characteristics of the guidewire 10 at this transition may occur. As such, it may be desirable to provide a tapered and/or ramp-like transition between the outer surface and/or diameter of the proximal end 28 and the outer surface of the core wire 30. While in some cases, a polymeric material may be used to create a smooth tapered transition and/or ramp transition between the outer surface and/or diameter of the proximal end 28 and the outer surface of the core wire 30, this often takes an additional manufacturing step—at least one to attach the tubular member 20 to the core wire 30, and an additional step of creating the ramp-like structure. For example, the proximal end 28 of the tubular member 20 could be spot welded to the core wire 30, but an additional step would be required to create a ramp-like structure to remedy the abrupt transition in the outer diameter of the guidewire.

The use of filet welding techniques can provide a desired alternative. For example, referring back to FIG. 1, the use of a filet weld 44 can provide a transition between the outer surface and/or diameter of the proximal end 28 and the outer surface of the core wire 30, while at the same time can provide for connection of the tubular member 20 to the core wire 30. In at least some embodiments, the weld 44 can provide a robust connection without the need for the use of additional attachment techniques between the proximal end 28 of the tubular member and the outer surface of the core wire 30.

Referring again to FIG. 2, the tubular member 20 can be disposed about a portion of the core wire 30, and welding equipment 60 can be used to deposit weld metal and/or material adjacent the distal end 28 of the tubular member 20 and the outer surface of the core wire 30 to create a fillet weld 44 (FIG. 1). For example, the weld metal and/or material can be deposited in and/or adjacent to the interior angle and/or corner 43 defined between the outer surface of the core wire 30 and the proximal end 28 of the tubular member 20. In some cases, the weld metal and/or material used to create the weld is separate material added during the welding process. However, in some embodiments, the weld metal and/or material may simply include a portion of the tubular member 20 and/or core member 30 that is heated with weld energy and flowed to create the weld 44. The weld 44 can be created and/or can extend radially about at least a portion of the outer surface of the core 30 and/or radially along the proximal end 28 of the tubular member 20. In other words, the weld 44 may extend in a radial fashion at least partially about the longitudinal axis of the core 30 and/or tubular member 20, and in some cases, extends all the way about the longitudinal axis. One way that this may be achieved is to rotate the assembly (tubular member 20 and core wire 30) as the weld energy and/or material is applied in a predetermined manner such that the weld 44 is formed radially about the assembly. In the embodiment shown, the distal tip member 37 is already disposed on the guidewire 10, but it should be understood that in other embodiments, the tubular member 20 may be attached to the core wire with the weld 44 first prior to creating and/or attaching the distal tip 37.

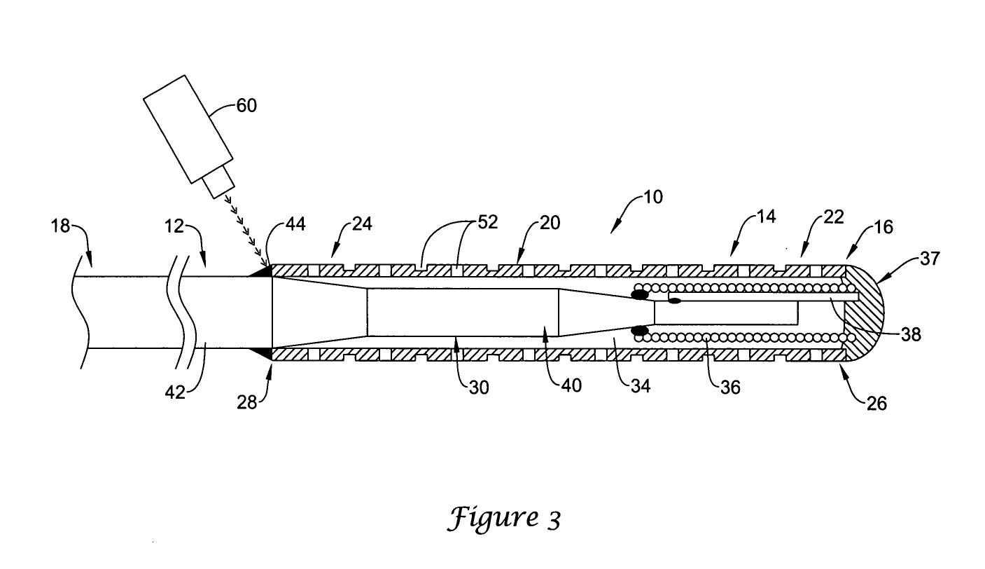

Refer now to FIG. 3, which shows the welding process nearing completion. As can be appreciated that as the weld 44 is created, it may fill a portion of and/or substantially all of the space in the interior angle and/or corner 43 defined between the proximal end 28 of the tubular member 20 and the outer surface of the core wire 30. As such, the weld 44 can attach the proximal end 28 of the tubular member 20 and the outer surface of the core wire 30, and create and/or exist as a transition or ramp-like structure between the outer surface of the tubular member 20 and the outer surface of the core wire 30.

The weld 44 can be created using any suitable welding techniques and/or equipment. Some examples of welding processes which may be suitable in some applications include LASER welding, resistance welding, TIG welding, microplasma welding, electron beam, and friction or inertia welding. Some examples of LASERs that may be suitable for LASER welding may include a Nd:YAG LASER, a CO₂ LASER, a Diode LASER, or the like, or others. LASER welding equipment which may be suitable in some applications is commercially available from Unitek Miyachi of Monrovia, Calif. and Rofin-Sinar Incorporated of Plymouth, Mich. Resistance welding equipment which may be suitable in some applications is commercially available from Palomar Products Incorporated of Carlsbad, Calif. and Polaris Electronics of Olathe, Kans. TIG welding equipment which may be suitable in some applications is commercially available from Weldlogic Incorporated of Newbury Park, Calif. Microplasma welding equipment which may be suitable in some applications is commercially available from Process Welding Systems Incorporated of Smyrna, Tenn.

In some example embodiments, the welding process is achieved by using a LASER welder, such as a Nd:YAG LASER. The core member 30 is disposed within the tubular member 20 such that the corner 43 is formed, and the LASER is directed at the corner 43. The LASER is set to pulse at a predetermined number of pulses per second, and the guidewire assembly is rotated at a given speed. The LASER is then activated. As the LASER hits the corner 43 (the proximal end 28 of the tube 20 and the adjacent outer surface of the core 30) it forms a fillet weld 44 around the entire circumference of the core 30 and tube 20. This joins the tube 20 to the core 30, and creates a smooth transition or ramp between the two structures. In some embodiments, the assembly can be rotated at a speed in the range of about 5 to about 15 RPM, and the LASER can be set to pulse in the range of about 1 to about 10 pulses per second, for a total number of pulses in the range of about 10 to about 50 total pulses.

As indicated above, the weld 44 may have a generally triangular and/or ramp like cross sectional shape and may join two surfaces (for example, the end surface of the tubular member 20 and the outer surface of the core wire 30) that meet in an interior angle. In some embodiments, the difference in size between the outer diameters of the proximal end of the tubular member 20 and the outer surface of the core wire 30 can be in the range of about 0.001 inch to about 0.2 inch, or in some embodiments, in the range of about 0.01 inch to about 0.08 inch. As such, the weld 44 may have a leg extending along the proximal end surface of the tubular member 20 that is in the range of about 0.01 inch to about 0.2 inch, or in some embodiments, in the range of about 0.01 inch to about 0.08 inch. Further, the weld 44 may include a leg that extends along the outer surface of the core wire 30 (in other words, the length of the weld as it extends along the longitudinal axis of the core wire) that is in the range of about 0.001 inch to about 0.2 inch, or in some embodiments, in the range of about 0.003 inch to about 0.03 inch. The tapered leg of the weld (for example, the leg that may be generally characterized as the hypotenuse of the generally triangular shaped weld) may have a length in the range of about 0.001 inch to about 0.2 inch, or in some embodiments, in the range of about 0.003 to about 0.03 inch. It should be understood, however, that there dimensions are by way of example only, and that a broad variety of other dimensions may be used.

As indicated above, the weld 44 can penetrate and fuse with the base metals of the core member 30 and the tubular member 20 to form the joint. The degree of penetration may be any suitable amount given the desired quality of the weld. In some embodiments, the degree of penetration within the material of the tubular member may be in the range of about 5% to about 100%, and the degree of penetration within the material of the core wire may be in the range of about 5% to about 100%.

It should also be understood that additional attachment points between the tubular member 20 and the core member 30 and/or other components of the guidewire 10 may be provided using any suitable attachment techniques, including any of those disclosed herein. Such additional attachment can be made in any suitable manner and at any suitable location, as desired and/or necessary. For example, the tubular member 20 may be connected to the core member 30, coil 36 and/or shaping ribbon 37 through the use of a solder tip 37. Those of skill in the art and others, however, will recognize that any of a broad variety of attachment techniques and/or structures may be used.

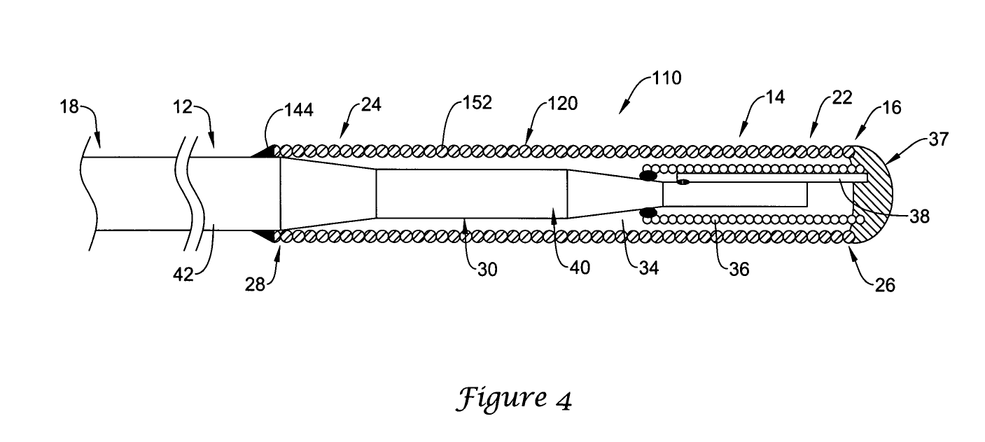

Another embodiment is shown in FIG. 4, wherein common reference numerals can refer to similar structure to the embodiments discussed above. In this embodiment, however, the tubular member is a coil member 120 disposed about the core member 30, and the proximal end 20 of the coil member 120 is welded to the core member 30, for example, with fillet weld 144. As can be appreciated, the filled weld 144 can be made in a similar fashion and/or include the similar structure and materials as weld 44 discussed above. As such, it can be appreciated that any of a broad variety of tubular member structures, such as a tubular member 20 or a coil 120, or the like, that may have outer diameter larger than an outer diameter of a core member 30 may be attached to the core member 30 through the use of a fillet weld 44/144 that can provide for a smooth transition and/or ramp-like structure at the joint.

A wide variety of materials and alternative features can also be used with any of the embodiments described herein. A description of some of these materials and alternative features with respect to at least some of the embodiments discussed above is given below. However, it should also be understood that any of these materials and/or alternative features can also be incorporated into any of the other embodiments described herein. The materials that can be used for the various components of guidewire 10 may include any that would serve the intended purpose and/or function. For example, core member 30, tubular member 20, coils 36 and 120, and/or shaping ribbon 38 may be made from a metal, metal alloy, a metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, a nickel-based alloy, such as a hastelloy, a nickel-cobalt based alloy, such as MP35-N, a nickel-copper based alloy, such as monel 400, a nickel-chromium based alloy, such as inconel 625, other Co—Cr alloys, platinum enriched stainless steel; or the like; or other suitable material.

Within the family of commercially available nickel-titanium or nitinol alloys, is a category designated “linear elastic” which, although it may be similar in chemistry to conventional shape memory and superelastic varieties, exhibits distinct and useful mechanical properties. By the applications of cold work, directional stress, and heat treatment, the material is fabricated in such a way that it does not display a substantial “superelastic plateau” or “flag region” in its stress/strain curve. Instead, as recoverable strain increases, the stress continues to increase in a generally linear relationship (as compared to that of super-elastic material, which has a super-elastic plateau) until plastic deformation begins. In some embodiments, the linear elastic nickel-titanium alloy is an alloy that does not show any substantial martensite/austenite phase changes that are detectable by DSC and DMTA analysis over a large temperature range.

For example, in some embodiments, there are no substantial martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “pushability” around tortuous anatomy. Accordingly, components of guidewire 10, such as core member 30 and/or tubular member 20, or others, may include or be made of linear elastic nickel-titanium alloy.

In some embodiments, the linear elastic nickel-titanium alloy is in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. In some other embodiments, a superelastic alloy, for example superelastic Nitinol can be used to achieve desired properties.

In one example, both the tubular member 20 and the core member 30 may comprise a nickel titanium alloy. In some other example embodiments, one of the tubular member 20 or the core member 30 may comprise stainless steel, and the other of the tubular member 20 or the core member 30 may comprise a nickel titanium alloy. In yet another example embodiment, the core member 30 can have a proximal section comprising stainless steel and a distal section comprising a nickel titanium alloy, and the tubular member 20 can comprise a nickel titanium alloy. As can be appreciated, these specific configurations are given by way of example, and that a broad variety of different configurations may be used including any of the materials listed herein, or others.

In at least some embodiments, portions or all of core member 30, tubular member 20, coils 36 and 120, and/or shaping ribbon 38, or other components that are part of or used in the device, may be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of guidewire 10 to achieve the same result.

In some embodiments, a degree of MRI compatibility is imparted into device 10. For example, to enhance compatibility with Magnetic Resonance Imaging (MRI) machines, it may be desirable to make core member 30, tubular member 20, coils 36 and 120, and/or shaping ribbon 38, or other portions of the medical device 10, in a manner that would impart a degree of MRI compatibility. For example, core member 30 and/or tubular member 20, or portions thereof, may be made of a material that does not substantially distort the image and create substantial artifacts (artifacts are gaps in the image). Certain ferromagnetic materials, for example, may not be suitable because they may create artifacts in an MRI image. Core member 30, tubular member 20, coils 36 and 120, and/or shaping ribbon 38, or portions thereof, may also be made from a material that the MRI machine can image. Some materials that exhibit these characteristics include, for example, tungsten, Elgiloy, MP35N, nitinol, and the like, and others.

Referring now to the tubular member 20 as in the embodiments shown in FIGS. 1-3, the tubular member 20 may include both a distal section 22 and a proximal section 24. In some embodiments the tubular member 20 can be a monolithic, single and/or a one-piece structure that defines both the proximal and distal ends 22/24. The tubular structure can also be a continuous and/or uninterrupted tubular member that defines both the proximal and distal sections 22/24. In other embodiments, the tubular member 20 may include a plurality of discrete tubular components or sections that are attached to one another to form the tubular member 20, or portions thereof. For example, the distal section 22 and proximal section 24 may each be a discrete tubular component that are attached and/or secured together to create the tubular member 20. In such a case, the components may be attached using any suitable joining or bonding technique and/or structure. For example, the distal and proximal sections 22/24 may be joined using adhesive bonding, welding, soldering, brazing, mechanical bonding and/or fitting, or the like, or any other suitable technique.

In some embodiments, as shown in FIGS. 1-3, the outer diameter of the tubular member 20 can be the same or substantially the same along the entire length of the tubular member 20. In other embodiments, the outer diameter of the tubular member proximal section 24 and the outer diameter of the tubular member distal portion 22 can be different. For example, the outer diameter of the tubular member proximal section 24 could be smaller than the outer diameter of the tubular member distal section 22. The change in diameter can be a sharp change in the diameter, it could be step-wise, or it could be a gradual change over a length of the tubular member 20. For example, the diameter of the tubular member 20 can gradually taper along some or all of the length of the tubular member 20, or along some or all of a proximal portion of the tubular member 20.

In embodiments where the distal and proximal sections 22/24 are two discrete and/or separate components that are attached, the variances in the outer diameters can be provided by the use of different discrete tubular components having different outer diameters. In embodiments where the tubular member 20 is a one-piece or monolithic member, the variances in the outer diameters can be provided by grinding or otherwise working the tubular member 20 to provide the desired diameters.

The tubular member 20 can optionally include a plurality of cuts, apertures, and/or slots 52 defined therein. In some embodiments, at least some, if not all of the slots 52 are disposed at the same or a similar angle with respect to the longitudinal axis of the tubular member 20. As shown, the slots 52 can be disposed at an angle that is perpendicular, or substantially perpendicular, to the tubular member longitudinal axis of the tubular member 20. However, in other embodiments, a group of one or more slots 52 may be disposed at different angles relative to another group of one or more slots 52.

The slots 52 may be provided to enhance the flexibility of the tubular member 20 while still allowing for suitable torque transmission characteristics. The slots or apertures 52 may be formed such that one or more rings and/or turns interconnected by one or more beams are formed in the tubular member 20, and such rings and beams may include portions of the tubular member 20 that remain after the slots 52 are formed in the body of the tubular member 20. Such an interconnected ring structure may act to maintain a relatively high degree of tortional stiffness, while maintaining a desired level of lateral flexibility. In some embodiments, some adjacent slots 52 can be formed such that they include portions that overlap with each other about the circumference of the tube 20. In other embodiments, some adjacent slots 52 can be disposed such that they do not necessarily overlap with each other, but are disposed in a pattern that provides the desired degree of lateral flexibility.

Additionally, the slots 52 can be arranged along the length of, or about the circumference of, the tubular member 20 to achieve desired properties. For example, the slots 52 can be arranged in a symmetrical pattern, such as being disposed essentially equally on opposite sides about the circumference of the tubular member 20, or equally spaced along the length of the proximal section 24 of the tubular member 20, or can be arranged in an increasing or decreasing density pattern, or can be arranged in a non-symmetric or irregular pattern. Other characteristics, such as slot size, slot shape and/or slot angle with respect to the longitudinal axis of the tubular member 20, can also be varied along the length of the tubular member 20 in order to vary the flexibility or other properties. In other embodiments, moreover, it is contemplated that the tubular member proximal section 24, or the entire tubular member 20, may not include any such slots 52.

Any of the above mentioned slots can be formed in essentially any known way. For example, slots 52 can be formed by methods such as micro-machining, saw-cutting, laser cutting, grinding, milling, casting, molding, chemically etching or treating, or other known methods, and the like. In some such embodiments, the structure of the tubular member 20 is formed by cutting and/or removing portions of the tube to form slots 52. Some example embodiments of appropriate micromachining methods and other cutting methods, and structures for tubular members and medical devices including tubular members are disclosed in U.S. Pat. Publication Nos. US 2003/0069522; and US 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference.

Forming the tubular member 20, or sections thereof, may include any one of a number of different techniques. For example, the tubular member 20, including the distal and proximal sections 22/24 and/or components, may be created by casting or forming methods, stamping methods, or the like, and may be shaped or otherwise worked, for example, by centerless grinding methods, into the desired shape and/or form. A centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing tubular member 20 during the grinding process. In some embodiments, tubular member 20 is centerless ground using a Royal Master HI-AC centerless grinder.

In the embodiment of FIG. 4, however, the tubular member different, in that it is a coil 120. The coil 120 may be formed of round wire or flat ribbon ranging in dimensions to achieve the desired flexibility. It can also be appreciated that other cross-sectional shapes or combinations of shapes may be utilized without departing from the spirit of the invention. For example, the cross-sectional shape of wires or filaments used to make the coil may be oval, rectangular, square, triangle, polygonal, and the like, or any suitable shape.

The coil 120 can be wrapped in a helical fashion by conventional winding techniques. The pitch of adjacent turns of coil 120 may be tightly wrapped so that each turn touches the succeeding turn or the pitch may be set such that coil 120 is wrapped in an open fashion. In some embodiments, the coil can have a pitch of up to about 0.04 inches, in some embodiments a pitch of up to about 0.02 inches, and in some embodiments, a pitch in the range of about 0.001 to about 0.004 inches. The pitch can be constant throughout the length of the coil 120, or can vary, depending upon the desired characteristics, for example flexibility. These changes in coil pitch can be achieved during the initial winding of the wire, or can be achieved by manipulating the coil after winding or after attachment to the guidewire. For example, in some embodiments, after winding of the coil 120, a larger pitch can be achieved on the distal portion of the coil 120 by simply pulling the coil. Additionally, in some embodiments, portions or all of the coil 120 can include coil windings that are pre-tensioned or pre-loaded during wrapping, such that each adjacent coil winding is biased against the other adjacent coil windings to form a tight wrap. Such preloading could be imparted over portions of, or over the entire length of the coil 120. The diameter of the coil 120 is preferably sized to fit around the core member 30, and to give the desired characteristics.

Referring now to core member 30, for example in each of the FIGS. 1-4, the entire core member 30 can be made of the same material along its length, or in some embodiments, can include portions or sections made of different materials. In some embodiments, the material used to construct core member 30 is chosen to impart varying flexibility and stiffness characteristics to different portions of core member 30. For example, the proximal region and the distal region of core member 30 may be formed of different materials, for example materials having different moduli of elasticity, resulting in a difference in flexibility. In some embodiments, the material used to construct the proximal region can be relatively stiff for pushability and torqueability, and the material used to construct the distal region can be relatively flexible by comparison for better lateral trackability and steerability. For example, the proximal region can be formed of straightened 304v stainless steel wire or ribbon and the distal region can be formed of a straightened super elastic or linear elastic alloy, for example a nickel-titanium alloy wire or ribbon.

In embodiments where different portions of core member 30 are made of different materials, the different portions can be connected using any suitable connecting techniques. For example, the different portions of core member 30 can be connected using welding (including laser welding), soldering, brazing, adhesive, or the like, or combinations thereof. Additionally, some embodiments can include one or more mechanical connectors or connector assemblies to connect the different portions of core member 30 that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. Nos. 09/972,276 (U.S. Pat. Publication No. 2003/0069520), 10/086,992 (U.S. Pat. Publication No. 2003/0069521, and 10/375,766 (U.S. Pat. Publication No. 2004/0167441), which are incorporated herein by reference.

Core member 30 can have a solid cross-section, for example a core wire, but in some embodiments, can have a hollow cross-section. In yet other embodiments, core member 30 can include a combination of areas having solid cross-sections and hollow cross sections. Moreover, core member 30, or portions thereof, can be made of rounded wire, flattened ribbon, or other such structures having various cross-sectional geometries. The cross-sectional geometries along the length of core member 30 can also be constant or can vary. For example, FIGS. 1-4 depict core member 30 as having a round cross-sectional shape. It can be appreciated that other cross-sectional shapes or combinations of shapes may be utilized without departing from the spirit of the invention. For example, the cross-sectional shape of core member 30 may be oval, rectangular, square, polygonal, and the like, or any suitable shape. Additionally, the core member 30 may include one or more tapered portions, for example, to provide for desired flexibility characteristics. Such tapers can be made or exist in a linear, stepwise, curvilinear, or other suitable fashion to achieve the desired results. For example, in the embodiment shown in FIGS. 1-4, the core member 30 includes a plurality of tapered sections and constant diameter sections.

In some embodiments, a sheath and/or coating, for example a lubricious, a hydrophilic, a protective, or other type of material may be applied over portions or all of the core member 30 and/or tubular member 20 or 120, or other portions of device 10. Some examples of suitable polymer sheath materials may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments sheath material can be blended with a liquid crystal polymer (LCP). For example, the mixture can contain up to about 6% LCP. This has been found to enhance torqueability. By employing selection of materials and processing techniques, thermoplastic, solvent soluble, and thermosetting variants of these and other materials can be employed to achieve the desired results. Some examples of suitable coating materials may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Some coating polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. Some examples of coatings would be disposing a coating on the thread member(s) and/or all or a portion of the tubular member and/or all or a portion of the core member.

A coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

The length of the guidewire 10 is typically dictated by the length and flexibility characteristics desired in the final medical device. For example, proximal section 12 may have a length in the range of about 20 to about 300 centimeters or more, the distal section 14 may have a length in the range of about 3 to about 50 centimeters or more, and the medical device 10 may have a total length in the range of about 25 to about 350 centimeters or more. It can be appreciated that alterations in the length of sections and/or of the guidewire 10 as a whole can be made without departing from the spirit of the invention.

It should also be understood that a broad variety of other structures and/or components may be used in the guidewire construction. Some examples of other structures that may be used in the guidewire 10 include one or more coil members, braids, shaping or safety structures, such as a shaping ribbon or wire, marker members, such as marker bands or coils, centering structures for centering the core wire within the tubular member, such as a centering ring, an extension system, for example, to effectively lengthen the guidewire for aiding in exchanging other devices, or the like, or other structures. Those of skill in the art and others will recognize that the materials, structure, and dimensions of the guidewire may be dictated primary by the desired characteristics and function of the final guidewire, and that any of a broad range of materials, structures, and dimensions can be used.

In a further embodiment, any of the tubular members described herein can also be incorporated into devices other than the guidewires that have been shown. As one example, any of the tubular members mentioned herein can be incorporated into a catheter shaft. In some cases, incorporating such tubular members into a catheter shaft can provide certain desirable characteristics, such as torque transmission and lateral flexibility, and the like. For example, a catheter shaft with a metallic tubular member filet welded to an inner tubular member may provide some for a good connection between the members, and may provide for a desirable transition in outer diameters.

It should be understood that this disclosure is, in many respects, only illustrative. Changes may be made in details, particularly in matters of shape, size, and arrangement of steps without exceeding the scope of the invention. For example, although set forth with specific reference to guidewires in some of the example embodiments shown in the Figures and discussed above, the invention may relate to virtually any medical device including an elongate metallic tubular member filet welded to a core structure and/or member. Thus, while the Figures and descriptions above are directed toward a guidewire, in other applications, sizes in terms of diameter, width, and length may vary widely, depending upon the desired properties of a particular device. The scope of the invention is, of course, defined in the language in which the appended claims are expressed.

What is claimed is:

1. A medical device comprising:
an elongated metallic tubular member defining an inner lumen and including an end;
a metallic core member including a first portion disposed within the lumen of the tubular member, and a second portion extending from the end of the tubular member, the core member including an outer surface, wherein the end of the metallic tubular member is attached to the outer surface of the core member with a fillet weld.

2. The medical device of claim 1, wherein the tubular member includes a tubular body including a plurality of slots formed therein.

3. The medical device of claim 1, wherein the tubular member comprises a coil member.

4. The medical device of claim 1, wherein the core member extends along a longitudinal axis, and the filet weld extends substantially around the longitudinal axis.

5. The medical device of claim 1, wherein the core member includes a first outer diameter, and the tubular member includes a second outer diameter that is greater than the first outer diameter, and wherein the fillet weld provides a tapered transition from the first outer diameter to the second outer diameter.

6. The medical device of claim 1, wherein the tubular member comprises a nickel titanium alloy.

7. The medical device of claim 1, wherein the core member comprises a nickel titanium alloy.

8. The medical device of claim 1, wherein the tubular member comprises stainless steel, platinum, or a nickel-cobalt based alloy.

9. The medical device of claim 1, wherein the core member comprises stainless steel, platinum, or a nickel-cobalt based alloy.

10. The medical device of claim 1, wherein the end of the tubular member is a proximal end of the tubular member, and wherein the first portion of the core member comprises a distal portion disposed within the lumen of the tubular member, and the second portion is a proximal portion extending proximally from the proximal end of the tubular member.

11. The medical device of claim 1, wherein the tubular member includes a distal end, and the device further includes a distal tip member disposed on the distal end of the metallic tubular member.

12. The medical device of claim 1, wherein the device comprises a guidewire.

13. A medical device comprising:
an elongated metallic tubular member defining an inner lumen and including a distal end and a proximal end including an outer diameter;
a metallic core member including a distal portion disposed within the lumen of the tubular member, and a proximal portion extending proximally from the tubular member, the proximal portion including an outer surface and having at least a section with an outer diameter that is less than the outer diameter of the tubular member;
a weld attaching the proximal end of the tubular member to the outer surface of the core member, the weld having a generally triangular cross-sectional shape.

14. The medical device of claim 13, wherein the weld is a fillet weld.

15. The medical device of claim 13, wherein the weld provides a tapered transition from the outer diameter of the proximal end of the tubular member to the outer diameter of the section of the core member.

16. A medical device comprising:
an elongated metallic tubular member defining an inner lumen and including an end having an outer diameter;
a metallic core member including a first portion disposed within the lumen of the tubular member, and a second portion extending from the end of the tubular member, the core member including an outer surface having at least a section with an outer diameter that is less than the outer diameter of the end of the tubular member such that a corner having an interior angle is formed between the end of the tubular member and the outer surface of the core member;
a weld metal deposited in the corner, the weld metal joining the tubular member and the core member.

17. The medical device of claim 16, wherein the weld metal forms a weld having a generally triangular cross-sectional shape.

18. The medical device of claim 16, wherein the weld provides a tapered transition from the outer diameter of the end of the tubular member to the outer diameter of the section of the core member.

19. A method of making a medical device, the method comprising:
providing an elongated metallic tubular member defining an inner lumen and including an end;
providing a metallic core member having an outer surface;
disposing a first portion of the metallic core member within the lumen of the tubular member such that a second portion of the core member extends from the end of the tubular member;
fillet welding the end of the tubular member to the outer surface of the core member.

20. A method of making a medical device, the method comprising:
providing an elongated metallic tubular member defining an inner lumen and including an end;
providing a metallic core member having an outer surface;
disposing a first portion of the metallic core member within the lumen of the tubular member such that a second portion of the core member extends from the end of the tubular member, the outer surface of the core member having at least a section with an outer diameter that is less than the outer diameter of the tubular member such that a corner having an interior angle is formed between the end of the tubular member and the outer surface of the core member;
depositing a weld metal in the corner, the weld metal joining the tubular member and the core member.

21. The method of claim 20, wherein depositing the weld metal in the corner includes forming a weld having a generally triangular cross-sectional shape.

22. The method of claim 20, wherein depositing the weld metal in the corner includes forming a weld that provides a tapered transition from the outer diameter of the end of the tubular member to the outer diameter of the section of the core member.
